(19) United States
(12) Patent Application Publication
(10) Pub. No.: US 2021/0106509 A1
(43) Pub. Date: Apr. 15, 2021

(54) A sunscreen composition

(71) Applicant: Conopco, Inc., d/b/a UNILEVER, Englewood Cliffs, NJ (US)

(72) Inventors: Rajkumar PERUMAL, Erode (Dist) (IN); Ashish Anant VAIDYA, Bangalore (IN)

(73) Assignee: Conopco, Inc., d/b/a UNILEVER, Englewood Cliffs, NJ (US)

(21) Appl. No.: 17/047,857

(22) PCT Filed: Apr. 15, 2019
(86) PCT No.: PCT/EP2019/059662

§ 371 Date: Oct. 15, 2020

(30) Foreign Application Priority Data
May 25, 2010 (IN) 1610/MUM/2010
Jul. 20, 2010 (IN) 2062/MUM/2010

(51) Int. Cl. A61K 8/34; A61Q 17/04

(52) U.S. Cl. CPC A61K 8/347; A61K 45/06; A61Q 17/04

(57) ABSTRACT

The present invention relates to a sunscreen composition. Particularly, the sunscreen composition relates to providing improved ultraviolet A protection factor (UVAPF). The composition comprises UVA organic sunscreen, 2-ethylhexyl 2-cyano-3,3-diphenylacrylate, a compound selected from resorcinol, phenylethyl resorcinol, 4-alkyl substituted resorcinol and mixtures thereof, fatty acid and soap.

### FIELD OF THE INVENTION

The present invention relates to a sunscreen composition. Particularly, the present invention relates to a sunscreen composition that provides improved ultraviolet (UV) A protection factor (UVAPF).

### BACKGROUND OF THE INVENTION

People often try to take care of themselves and of their external surfaces e.g. skin and scalp including hairs, with a desire of enjoying healthy lifestyle.

Specific skin related issues that people are concerned about include healthy and infection-free skin, good skin tone, adequate moisturization and protection from UV radiation contained in the sunlight.

Sunlight comprises infrared region (700 nm to 1 mm), visible region (380 to 780 nm) and UV region (100 to 400 nm). The UV region is composed of UVC (100 to 280 nm), UVB (280 to 315 nm) and UVA (315 to 400 nm). While the human body needs some UV radiation for synthesis/maintenance of adequate amount of vitamin D, harmful effects of UV typically outweighs the benefits. An excessive exposure to UVB is said to cause direct damage to DNA and cause sunburn too; and an excessive exposure to UVA is said to give rise to immediate pigment darkening and delayed tanning effect. In addition, UVA is also said to cause DNA damages via oxidation reactions that involve reactive oxygen species (Zhang et al., 1997, Free Radical Biology & Medicine, 23, 980-985).

It is for reasons like these that people are inclined towards avoiding exposure to sunlight as much as possible. However, in many instances, avoiding exposure to sunlight is very difficult and at times; impossible. It is for such unavoidable reasons, people tend to make use of various topical compositions e.g. sunscreen composition, that offer at least some protection against unwanted effects caused by excessive exposure to UV radiations contained in sunlight. These compositions comprise sunscreens that absorb UV radiations and prevent them from reaching a surface e.g. skin of a user on which the composition is applied.

Organic sunscreens e.g. 4-tert-butyl-4′-methoxydibenzoylmethane (BMDM) absorb the UVA radiation. The ability of a sunscreen to provide protection by absorbing UVA radiation is expressed in terms of UVA protection factor (UVAPF). On the other hand, organic sunscreens, e.g. 2-ethylhexyl 2-cyano-3,3-diphenylacrylate (OCR) and octyl methoxycinnamate (MCX) absorb the UVB radiation. The ability of a sunscreen to provide protection by absorbing UVB radiation is expressed in terms of sun protection factor (SPF).

EP 2 431 021 (Unilever) discloses a high SPF sunscreen composition comprising less than 8% total organic sunscreens comprising dibenzoylmethane or its derivative, an oil soluble UV-B organic sunscreen, non-ionic surfactant selected from the class of fatty alcohol ethoxylates with saturated carbon chain and having HLB higher than 15.5 or from the class of fatty alcohol ethoxylates with unsaturated carbon chain with HLB higher than 12, a polymer of the class of acrylate/R-methacrylate copolymer; or an acrylate/R-alkyl acrylate crosspolymer and a cosmetically acceptable base. The compositions disclosed therein aim at providing a SPF higher than 15 without compromising on the desired skin sensorial properties.

WO 03/080009 (Unilever) discloses cosmetic compositions comprising organic sunscreens along with a 4-substituted resorcinol derivatives in a cosmetically acceptable base. The compositions exhibit improved storage stability and oxidative stability. WO 03/080009 particularly discloses a combination of butylmethoxy dibenzoylmethane, octyl methoxycinnamate, stearic acid and 4-substituted resorcinol.

WO 11/147738 (Unilever) relates to sunscreen compositions that provide SPF (equal to or greater than 15) and high UVAPF (equal to or higher than 4). WO 11/147738 discloses a solid sunscreen composition comprising butylmethoxy dibenzoylmethane, a nonionic surfactant of select class, a polymer of select class in a cosmetically acceptable base. WO 11/147738 further discloses a non-solid sunscreen composition comprising dibenzoylmethane or its derivative, a nonionic surfactant of select class, a fatty acid and a cosmetically acceptable base.

WO 16/142129 (Unilever) discloses a combination of C12-C20 fatty acid with C12-C20 fatty acid soap, white pigment e.g. titanium dioxide in a cosmetically acceptable carrier wherein the mole ratio of soap to fatty acid is from 0.23 to 0.5. WO 16/142129 solves the problem of providing photoprotection whilst maintaining the sensorial properties of the compositions when pigments like titanium dioxide are added to fatty acid containing compositions.

Despite efforts thus far, compositions that provide improved UVAPF remains a topic of interest.

Need therefore exists to develop a sunscreen composition that provides improved UVAPF.

It has now been found that a composition comprising a compound selected from resorcinol, phenylethyl resorcinol, 4-alkyl substituted resorcinol and mixtures thereof along with UVA organic sunscreen, UVB organic sunscreen of select class, fatty acid and soap, provides improved UVAPF.

### SUMMARY OF THE INVENTION

In a first aspect, the present invention relates to a sunscreen composition comprising:
a. 0.1 to 5 wt % UVA organic sunscreen,
b. 0.1 to 10 wt % 2-ethylhexyl 2-cyano-3,3-diphenylacrylate,
c. 0.1 to 2.5 wt % of a compound selected from resorcinol, phenylethyl resorcinol, 4-alkyl substituted resorcinol and mixtures thereof,
d. 4 to 25 wt % fatty acid; and
e. 0.1 to 10 wt % soap.

In a second aspect, the present invention relates to a method of providing improved UVAPF, the method comprising applying on the skin a sunscreen composition of the first aspect and optionally, rinsing with water.

In a third aspect, the present invention relates to use of the composition of the first aspect for providing improved UVAPF.

### DETAILED DESCRIPTION OF THE INVENTION

Any feature of one aspect of the present invention may be utilized in any other aspect of the invention. The word “comprising” is intended to mean “including” but not necessarily “consisting of” or “composed of.” In other words, the listed steps or options need not be exhaustive. Except in the operating and comparative examples, or where otherwise explicitly indicated, all numbers in this description indicating amounts of material or conditions of reaction, physical properties of materials and/or use are to be understood as modified by the word “about”. Numerical ranges expressed in the format “x to y” are understood to include x and y. When for a specific feature multiple preferred ranges are described in the format “x to y”, it is understood that all ranges combining the different endpoints are also contemplated.

Unless specified otherwise, amounts as used herein are expressed in percentage by weight based on total weight of the composition and is abbreviated as “wt %”.

The use of any and all examples or exemplary language e.g. “such as” provided herein is intended merely to better illuminate the invention and does not in any way limit the scope of the invention otherwise claimed.

In a first aspect, the present invention relates to a sunscreen composition comprising:
a. 0.1 to 5 wt % UVA organic sunscreen,
b. 0.1 to 10 wt % 2-ethylhexyl 2-cyano-3,3-diphenylacrylate,
c. 0.1 to 2.5 wt % of a compound selected from resorcinol, phenylethyl resorcinol, 4-alkyl substituted resorcinol and mixtures thereof,
d. 4 to 25 wt % fatty acid; and
e. 0.1 to 10 wt % soap.

UVA Organic Sunscreen

The composition according to the present invention (the composition) comprises UVA organic sunscreen that absorbs UVA radiations and prevents them from reaching a surface e.g. skin of a user.

Examples of sunscreens that may be used as UVA organic sunscreen in the composition are dibenzoylmethane compound, bisdisulizole disodium (commercially available as Neo Heliopan® AP), diethylamino hydroxybenzoyl hexyl benzoate (commercially available as Uvinul® A Plus), Ecamsule (commercially available as Mexoryl SX) and Methyl anthranilate.

Preferably, sunscreen that may be used as UVA organic sunscreen is selected from a dibenzoylmethane compound, diethylamino hydroxybenzoyl hexyl benzoate, and mixtures thereof.

More preferably, sunscreen that may be used as UVA organic sunscreen is selected from BMDM (commercially available as Parsol® 1789 or Avobenzone), 2-methyldibenzoylmethane, 4-isopropyldibenzoyl-methane, 4-tert-butyldibenzoylmethane, 2,4-dimethyldibenzoylmethane, 2,5-dimethyldibenzoylmethane, 4,4′-diisopropyl-dibenzoylmethane, 2-methyl-5-isopropyl-4′-methoxydibenzoylmethane, 2-methyl-5-tert-butyl-4′-methoxy-dibenzoyl methane, 2,4-dimethyl-4′-methoxy dibenzoylmethane, 2,6-dimethyl-4-tert-butyl-4′-methoxy-dibenzoylmethane and mixtures thereof.

Most preferably, sunscreen that may be used as UVA organic sunscreen is BMDM.

The composition comprises 0.1 to 5 wt %, preferably 0.5 to 4.5 wt %, more preferably 1 to 4 wt %, even more preferably 1 to 3.5 wt %, further more preferably 1 to 3 wt %, still more preferably 1 to 2.5 wt % of one or more UVA organic sunscreens.

### 2-ethylhexyl 2-cyano-3,3-diphenylacrylate (OCR)

The composition comprises OCR (commercially available as Octocrylene™) that absorbs UVB radiation and prevents them from reaching a surface e.g. skin.

The composition comprises 0.1 to 10 wt %, preferably 0.5 to 9 wt %, more preferably 1 to 8 wt %, even more preferably 1.5 to 7 wt %, further more preferably 2 to 6 wt %, still more preferably 2.5 to 5 wt % and most preferably 3 to 4 wt % OCR.

Resorcinol, Phenylethyl Resorcinol (PER), 4-Alkyl Substituted Resorcinol

The composition comprises a compound selected from resorcinol, PER, 4-alkyl substituted resorcinol and mixtures thereof. Such compounds are known to provide skin lightening effect.

Preferably, the compound selected is 4-alkyl substituted resorcinol. The alkyl group in 4-alkyl substituted resorcinol can be straight chain alkyl or branched chain alkyl. For example, the alkyl group can be straight chain alkyl as in the case of 4-propyl resorcinol or it can be a branched chain alkyl like as in the case of 4-isopropyl resorcinol (IPR).

Examples of 4-alkyl substituted resorcinol include 4-methyl resorcinol, 4-ethyl resorcinol (ER), 4-propyl resorcinol, IPR, 4-butyl resorcinol (BR), 4-pentyl resorcinol, 4-hexyl resorcinol (HR), 4-heptyl resorcinol, 4-octyl resorcinol and mixtures thereof.

Preferred 4-alkyl substituted resorcinol are ER, BR, HR and mixtures thereof. More preferred 4-alkyl substituted resorcinol are ER, HR and mixtures thereof.

It will be understood that the composition may comprise a combination of one or more compounds selected from resorcinol, PER and 4-alkyl substituted resorcinol. For example, the composition may comprise one or more 4-alkyl substituted resorcinol in presence or absence of resorcinol. Preferably, the composition comprises one compound selected from resorcinol, PER, 4-alkyl substituted resorcinol.

The composition comprises 0.1 to 2.5 wt %, preferably 0.1 to 2.25 wt %, more preferably 0.25 to 2 wt %, even more preferably 0.25 to 1.5 wt %, further more preferably 0.25 to 1 wt %, still more preferably 0.25 to 0.75 wt % and most preferably 0.25 to 0.5 wt % resorcinol, PER, 4-alkyl substituted resorcinol and mixtures thereof.

Fatty Acid

The composition comprises fatty acid. Fatty acids when present in a composition along with a soap provides the so called vanishing cream effect, i.e. a composition, when applied on to the human skin, vanishes on the skin leaving behind no significant streaks of the composition.

The composition comprises fatty acids having carbon atoms preferably in the range 10 to 22, more preferably 12 to 22, even more preferably 14 to 22, further more preferably 16 to 22, yet more preferably from 18 to 22. It is found that a fatty acid with increasing chain length provides increasing UVAPF and/or increasing SPF.

Examples of fatty acids that may be used in the composition include lauric, myristic, palmitic, stearic, isostearic, oleic, linoleic, arachidic, behenic, erucic acid and mixtures thereof.

Alternatively, the fatty acid that may preferably be used is stearic acid or palmitic acid or a mixture thereof. The fatty acid in the present invention is preferably hystric acid which is substantially (generally about 90 to 95%) a mixture of stearic acid and palmitic acid in a ratio of between 55:45 to 45:55.

The composition comprises 4 to 25 wt %, preferably 6 to 22 wt %, more preferably 8 to 20 wt %, even more preferably 10 to 19 wt % and further more preferably 12 to 18 wt % fatty acid.

Soap

The composition comprises soap. Soap when present in combination with fatty acid in the composition provides the so called vanishing effect.

Soap of the invention is generally prepared by in-situ neutralization of fatty acid present in the composition. Thus, it is preferred that the soap has a carbon chain length that corresponds to the chain length of fatty acid in the composition. The soap is formed from the fatty acid through use of alkali metal hydroxides e.g. sodium hydroxide or potassium hydroxide. Of the two, potassium hydroxide is more preferred. Thus, the soap is preferably a potassium soap (potassium salt of fatty acid).

The composition comprises 0.1 to 10 wt %, preferably 1 to 8 wt %, more preferably 2 to 7 wt %, even more preferably 2 to 6 wt %, further more preferably 2 to 5 wt % and most preferably 2 to 4 wt % soap.

Preferably, the composition further comprises a nonionic surfactant having HLB value in the range 9 to 20, preferably 10 to 19, more preferably 12 to 18, even more preferably 13 to 17 and yet more preferably 15 to 17.

HLB is calculated using the Griffin method wherein HLB=20×Mh/M wherein Mh is the molecular mass of the hydrophilic portion of the molecule and M is the molecular mass of the whole molecule, giving a result on an arbitrary scale of 0 to 20. Typical values for various surfactants are given below:

A value <10: Lipid soluble (water insoluble)

A value >10: Water soluble

A value from 4 to 8 indicates an anti-foaming agent

A value from 7 to 11 indicates a W/O (water in oil) emulsifier

A value from 12 to 16 indicates oil in water emulsifier

A value from 11 to 14 indicates a wetting agent

A value from 12 to 15 is typical of detergents

A value of 16 to 20 indicates a solubiliser or a hydrotrope.

Preferably, the nonionic surfactant having HLB value in the range 9 to 20 is selected from fatty alcohol ethoxylates, alkyl phenol ethoxylates, polyoxyethylene sorbitan alkyl esters and mixtures thereof. Preferably, the nonionic surfactants are ones with at least 9 alkylene oxide groups preferably at least 9 ethylene oxide groups.

Examples of fatty alcohol ethoxylates that may be used as nonionic surfactants in the composition include polyoxyethylene lauryl ether (HLB=16.9; commercially available as Brij® 35), polyoxyethylene (20) cetyl ether (HLB=16; commercially available as Brij® 58), polyethylene glycol octadecyl ether (HLB=18.8; commercially available as Brij® 700) and Laureth-9 (C12E09; HLB=14.3; commercially available as Brij® L9).

Examples of alkyl phenol ethoxylates that may be used as nonionic surfactant in the composition include octylphenol ethoxylate (HLB=15.5; commercially available as Triton™ X165), octylphenol ethoxylate (HLB=17.6; commercially available as Triton™ X405) and octylphenol ethoxylate (HLB=18.4; commercially available as Triton™ X705).

Examples of polyoxyethylene sorbitan alkyl esters that may be used as the nonionic surfactant in the composition include polyoxyethylenesorbitan monolaurate (HLB=13.3; commercially available as Tween® 21), polyoxyethylenesorbitan monolaurate (HLB=16.7; commercially available as Tween® 20), Polyoxyethylenesorbitan monopalmitate (HLB=15.6; commercially available as Tween® 40) and polyoxyethylene sorbitan monostearate (HLB=14.9; commercially available as Tween® 60).

More preferably, the nonionic surfactant having HLB value in the range 9 to 20 that may be present in the composition is fatty alcohol ethoxylate with saturated carbon chain having HLB higher than 15.5.

Preferably, the composition comprises 0.5 to 5 wt %, more preferably 1 to 4 wt %, even more preferably from 2 to 3 wt % nonionic surfactant having HLB in the range 9 to 20.

Preferably, the composition further comprises a polymer. The polymer acts as thickener in the composition and improves sensorial properties of the composition.

The polymer is preferably selected from the following classes:
acrylate/R-methacrylate copolymer e.g. acrylates/steareth-20 methacrylate copolymer (commercially available as Aculyn™ 22) and acrylates/beheneth-25 methacrylate copolymer (commercially available as Aculyn™ 28),
acrylate/R-methacrylate crosspolymer e.g. acrylates/steareth-20 methacrylate crosspolymer (commercially available as Aculyn™ 88),
acrylates copolymer (commercially available as Aculyn™ 33),
acrylate/R-alkyl acrylate crosspolymer e.g. acrylates/C10-C30 alkyl acrylate crosspolymer (commercially available as Pemulen™ TR-2),
copolymer of ammonium acryloyldimethyltaurate with vinyl pyrrolidone (commercially available as Aristoflex® AVC),
copolymer of sodium acryloyldimethyltaurate with vinyl pyrrolidone (commercially available as Aristoflex® AVS); and
crosspolymer of acryloyldimethyltaurate with R-alkyl acrylate and methyacrylate e.g. Ammonium acryloyldimethyltaurate/beheneth-25 methacrylate crosspolymer (commercially available as Aristoflex® HMB and Aristoflex® BLV); and mixtures thereof.

More preferably, the polymer is selected from acrylate/R-methacrylate copolymer, acrylates copolymer and mixtures thereof.

Preferably, the composition comprises 0.1 to 5 wt %, more preferably 0.5 to 4.5 wt %, even more preferably 1 to 4 wt %, further more preferably from 1.5 to 3.5 wt %, still more preferably from 2 to 3 wt % polymer.

Preferably, the composition comprises a cosmetically acceptable vehicle that includes water and may be present in the composition in amount 5 to 99.9 wt %, preferably 10 to 95 wt %, more preferably 15 to 90 wt %, even more preferably 20 to 80 wt %, further more preferably 25 to 75 wt % and still more preferably 30 to 70 wt %.

Preferably, the composition further comprises one or more skin lightening agents. These one or more skin lightening agents may be selected from niacinamide, vitamin B6, vitamin C, vitamin A, glutathione precursors, galardin, adapalene, aloe extract, ammonium lactate, arbutin, azelaic acid, butyl hydroxy anisole, butyl hydroxy toluene, citrate esters, deoxyarbutin, 1,3-diphenyl propane derivatives, 2,5-dihydroxybenzoic acid and its derivatives, 2-(4-acetoxyphenyl)-1,3-dithiane, 2-(4-hydroxyphenyl)-1,3-dithiane, ellagic acid, gluco pyranosyl-1-ascorbate, gluconic acid, glycolic acid, green tea extract, 4-Hydroxy-5-methyl-3[2H]-furanone, 4-hydroxyanisole and its derivatives, 4-hydroxybenzoic acid derivatives, hydroxycaprylic acid, inositol ascorbate, lactic acid, lemon extract, linoleic acid, magnesium ascorbyl phosphate, 5-octanoyl salicylic acid, salicylic acid, 3,4,5-trihydroxybenzyl derivatives, acetylglucosamine, pitera extract, symwhite, calcium pantothenate (Melano-block), seppiwhite, soybean extract (bowman birk inhibitor), 12-hydroxystearic acid and mixtures thereof. When used in the composition, 12-hydroxystearic acid is used as a skin lightening agent and not as a fatty acid.

Preferably, skin lightening agents that may be used in the composition are niacinamide, vitamin B6, 12-hydroxystearic acid, glutathione precursors, galardin and mixtures thereof.

When incorporated in the composition, a skin lightening agent may be added in an amount 0.001 to 15 wt %, preferably 0.01 to 10 wt %, more preferably 0.1 to 5 wt %, even more preferably 0.5 to 3 wt %, in the composition.

Preferably, the composition further comprises emollients. Examples of emollients that may be used in the composition include stearyl alcohol, glyceryl monoricinoleate, mink oil, cetyl alcohol, isopropyl isostearate, isobutyl palmitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, silicone oils such as dimethylpolysiloxane, din-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rape seed oil, safflower seed oil, evening primrose oil, soybean oil, sunflower seed oil, avocado oil, sesame seed oil, coconut oil, arachis oil, castor oil, acetylated lanolin alcohols, petroleum jelly, mineral oil, butyl myristate, isopropyl linoleate, lauryl lactate, myristyl lactate, decyl oleate, myristyl myristate and mixtures thereof.

Preferably, the composition further comprises solvents. Examples of solvents that may be used in the composition include ethyl alcohol, isopropanol, acetone, ethylene glycol monoethyl ether, diethylene glycol monobutyl ether, diethylene glycol monoethyl ether and mixtures thereof.

Preferably, the composition further comprises powders. Examples of powders that may be used in the composition include chalk, talc, fullers earth, kaolin, starch, gums, colloidal silica sodium polyacrylate, tetra alkyl and/or trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose, ethylene glycol monostearate and mixtures thereof.

Preferably, the composition further comprises preservatives to protect against the growth of potentially harmful microorganisms. Examples of ingredients that may be used as preservatives in the composition include alkyl esters of para-hydroxybenzoic acid, hydantoin derivatives, propionate salts, and a variety of quaternary ammonium compounds. More preferably, ingredients that may be used as preservative in the composition are sodium benzoate, iodopropynyl butyl carbamate, methylisothiazolinone, iodopropynylbutylcarbamate, phenoxyethanol, methyl paraben, propyl paraben, imidazolidinyl urea, sodium dehydroacetate, ethylhexylglycerin, benzyl alcohol, alkane diols and mixtures thereof. The alkane diols that are suitable for use as preservative are C₆-C₁₂ alkanes that are vicinally substituted with hydroxy groups. Illustrative examples include 1,2-octane diol (caprylyl glycol), 2,3-octane diol, 1,2-nonane diol, 1,2-decane diol, 1,2-hexane diol, 3,4-octane diol, mixtures thereof or the like where caprylyl glycol is typically the most preferred.

When present in the composition, preservatives are added preferably in an amount 0.001 to 5 wt %, more preferably 0.01 to 3 wt % and most preferably 0.02 to 2 wt %.

The composition may further comprise a range of other optional ingredients that include antioxidants, binders, biological additives, buffering agents, colorants, astringents, fragrance, opacifying agents, conditioners, exfoliating agents, pH adjusters, skin soothing agents, and skin healing agents.

The composition is preferably formulated in the form of a powder, flake, lotion, cream, gel or mousse. More preferably, the composition is formulated in the form of cream or lotion and most preferably in the form of cream. The composition can be a leave-on or wash-off type of composition. The composition is preferably a leave-on type of composition. The packaging for the composition of this invention can be a patch, bottle, tube, roll-ball applicator, propellant driven aerosol device, squeeze container or lidded jar.

It is found that a resorcinol, phenylethyl resorcinol, 4-alkyl substituted resorcinol and mixtures thereof improve the UVAPF when present in the composition according to the first aspect. It is also found that such improved UVAPF is obtained only when OCR was used in presence of UVA organic sunscreen. An improved UVAPF was not obtained when other UVB organic sunscreen e.g. MCX was used instead of OCR. OCR is known to stabilize dibenzoylmethane compound, e.g. BMDM. It is found that improvement in UVAPF obtained in presence of a compound selected from resorcinol, phenylethyl resorcinol, 4-alkyl substituted resorcinol and mixtures thereof, is over and above the UVAPF obtained when OCR is used instead of MCX in the same composition. Thus, it is surprising that replacing one UVB sunscreen, i.e. MCX, with another UVB sunscreen, i.e. OCR, has surprisingly resulted in improved UVAPF where delivering UVAPF is a function of UVA sunscreen.

Preferably, the sunscreen composition is a composition comprising:
a. 0.1 to 5 wt % dibenzoylmethane compound,
b. 0.1 to 10 wt % 2-ethylhexyl 2-cyano-3,3-diphenylacrylate,
c. 0.1 to 2.5 wt % a compound selected from BR, ER, HR and mixtures thereof,
d. 4 to 25 wt % fatty acid; and
e. 0.1 to 10 wt % soap.

More preferably, the sunscreen composition is a composition comprising:
a. 0.1 to 5 wt % 4-tert-butyl-4′-methoxydibenzoylmethane,
b. 0.1 to 10 wt % 2-ethylhexyl 2-cyano-3,3-diphenylacrylate,
c. 0.1 to 2.5 wt % a compound selected from ER, HR and mixtures thereof,
d. 4 to 25 wt % fatty acid; and
e. 0.1 to 10 wt % soap.

In a second aspect, the present invention further relates to a method of providing improved UVAPF, wherein the method comprises the step of:
a. applying on to the skin a sunscreen composition of the first aspect; and
b. optionally, rinsing with water.

The present invention also provides a method of providing improved UVAPF comprising the steps of applying the composition of the first aspect on to a surface e.g. skin, in case of a leave-on composition. This method optionally comprises an additional step of at least partially removing the composition from the surface if it is in the form of a wash-off composition. Preferably, the method is non-therapeutic or cosmetic. When the composition is applied on to the skin, an improved UVAPF is obtained.

In a third aspect, the present invention relates to use of the composition of the first aspect for improved UVAPF.

The present invention now will be demonstrated by way of following non-limiting examples.

### Examples

Protocols

Preparation of Composition

Briefly, the composition as per the present invention was prepared as follows: Demineralised water and potassium hydroxide were charged in a beaker (main beaker) mounted on a magnetic stirrer and heater. The water phase was heated to 75° C. and maintained at the same temperature. In a side pot, a fatty acid mentioned in examples below was heated to 75° C. for melting and was added to the main beaker maintained at 75° C. The system was stirred for 5 minutes at 75° C. The sunscreens as mentioned in examples, other oil soluble ingredients and nonionic surfactant were taken in a separate beaker and heated to 65 to 70° C. for melting. They were mixed well and were added to the main beaker. Stirring was continued for another 5 minutes at 75° C. The stirrer and heater were switched off and mixing was done manually until temperature reached ambient (25° C.).

Measurement of In-Vitro SPF/UVA-PF

Thin film transmittance measurements were done using SPF-290S SPF meter (Optometrics Corporation). In this study, 70×70 mm PMMA plate with 6 μm roughness from Schonberg GmbH & Co were used. The percent transmittance of the various compositions was measured as follows. 2 mg/cm² of a test sample was applied on PMMA plate, distributed uniformly using a syringe, and spread uniformly. The drying time for the PMMA plates was 30 minutes. After the drying time, sample plates were exposed to UV light and transmittance scan was recorded. This scan gives the transmittance as a function of wavelength (290 to 400 nm) for a given sample. For a single plate, six to eight different spots were scanned. The same was repeated for 3 plates. The data reported is thus an average over 18 readings. The reference transmittance scan was obtained using a blank plate, with no sample on the PMMA plates with glycerin spread on it. The transmittance values were used to arrive at the SPF values using the Win SPF software provided with the instrument. The transmittance values used to calculate the UVAPF values for each composition.

In-Vivo SPF/UVAPF

The compositions outlined in examples shown in Table 1 were tested in a randomized human volunteer study carried out with appropriate controls. Briefly, minimum erythemal dose (MED) and minimal persistent pigment darkening dose (MPPDD) for each volunteer was estimated before sample (compositions in table 1) application. The test samples were blinded with the standard samples for which the SPF/UVAPF is known. The required amount of sample (2 mg/cm²) was applied on the back skin of volunteers. The samples were dispensed uniformly using a syringe and applied uniformly using a finger cot over a period of 20 to 50 seconds which depends on the ease of application of a composition. For each sample new finger cot is used. Post sample application, the subjects were exposed to UV, dosage of which is calculated based on MED and MPPDD (specific to each volunteer) and on the anticipated or target SPF/UVAPF. Post exposure, volunteers were evaluated for the erythema and

MPPDD. An evaluation of the SPF of was carried out using ISO Standard 24444: 2010. An evaluation of UVAPF was carried out using ISO 24442:2011. The data reported here is an average of 5 volunteers.

TABLE 1

| Ingredients | 1 | 2 | A | B | C |
|---|---|---|---|---|---|
| Soap (formed in-situ) | 2.62 | 2.62 | 2.62 | 2.62 | 2.62 |
| Hystric acid | 17 | 17 | 17 | 17 | 17 |
| Nonionic surfactant | 2 | 2 | 2 | 2 | 2 |
| (Brij 35) | | | | | |
| UVA Sunscreen (BMDM) | 1.2 | 1.2 | 1.2 | 1.2 | 1.2 |
| 2-ethylhexyl 2-cyano- | 3 | 3 | 3 | — | — |
| 3,3-diphenylacrylate (OCR) | | | | | |
| MCX | — | — | — | 2.4 | 2.4 |
| HR | 0.5 | 0.25 | — | — | 0.5 |
| Polymer (Aculyn 22) | 1 | 1 | 1 | 1 | 1 |
| Polymer (Aculyn A33) | 0.5 | 0.5 | 0.5 | 0.5 | 0.5 |
| In-vivo UVAPF | 14 | 17 | 13 | 7 | 7 |

The data in table 1 shows that improved UVAPF is obtained using compositions as per the present invention (examples 1 and 2) that comprises HR along with BMDM, OCR, fatty acid and soap as compared to the comparative examples (example A and B; not as per the present invention) that do not contain HR.

Further, the data also shows that improved UVAPF is obtained only when a select UVB sunscreen, i.e. OCR (example 1), is used instead of MCX (example C) despite that both these compositions comprise the same amount, i.e. 0.5 wt %, of HR. Thus, HR provided improved UVAPF when used in combination with UVA organic sunscreen, 2-ethylhexyl 2-cyano-3,3-diphenylacrylate, fatty acid and soap. This improved UVAPF was not obtained when MCX was present instead of OCR.

The data in table 1 also shows that absence or presence of HR in the compositions of example B and C has no effect on UVAPF.

Surprisingly, it was found that improvement in UVAPF was not dependent on dose of HR used. As seen from example 2 (0.25 wt % HR), UVAPF equal to 17 was obtained whereas example 1 (0.5 wt % HR) showed UVAPF equal to 14. It is believed that such an effect is due to superoxide quenching efficacy which was found to increase with increasing concentration of HR up to a concentration (2 mM). Thereafter, superoxide quenching efficacy decreased despite increase in concentration of HR under uniform light exposure conditions.

Effect of Amount of Soap in the Composition on UVAPF and SPF

The compositions outlined in table 2 demonstrate the effect of presence of soap in the composition as compared to the control (no soap).

TABLE 2

| Ingredients | D | 3 | 4 | 5 |
|---|---|---|---|---|
| Soap (formed in-situ) | 0 | 2.62 | 5 | 7.5 |
| Hystric acid | 17 | 17 | 17 | 17 |
| Nonionic surfactant | 2 | 2 | 2 | 2 |
| (Brij 35) | | | | |
| UVA Sunscreen BMDM | 1.2 | 1.2 | 1.2 | 1.2 |
| OCR | 3.0 | 3.0 | 3.0 | 3.0 |
| MCX | — | — | — | — |
| HR | 0.25 | 0.25 | 0.25 | 0.25 |
| Polymer (Aculyn 22) | 1 | 1 | 1 | 1 |
| Polymer (Aculyn 33) | 0.5 | 0.5 | 0.5 | 0.5 |
| In-vitro SPF | 9.52 ± 0.78 | 22.04 ± 3.04 | 16.96 ± 1.58 | 15.75 ± 0.95 |
| In-vitro UVAPF | 6.87 ± 0.37 | 10.63 ± 0.83 | 9.28 ± 0.57 | 9.03 ± 0.40 |

The data in table 2 shows that soap, when present in the composition as per the present invention, boosts SPF as well as UVAPF.

Further, additional compositions as shown in table 3 below were prepared as described earlier and UVAPF and SPF were measured as described earlier. The results found were as provided in table 3 below.

TABLE 3

| Ingredients | 6 | 7 | 8 | 9 |
|---|---|---|---|---|
| Soap (formed in-situ) | 2.5 | 2.75 | 2.62 | 2.62 |
| Polymer (Aculyn 33) | 0.5 | 0.5 | 0.5 | 0.5 |
| Nonionic surfactant | 2 | 2 | 2 | 2 |
| (Brij 35) | | | | |
| OCR | 3 | 3 | 3 | 3 |
| Palmitic acid | 17 | — | — | — |
| Stearic acid | — | 17 | — | — |
| Hystric Acid | — | — | 17 | 17 |
| BMDM | 1.2 | 1.2 | 1.2 | 1.2 |
| HR | 0.25 | 0.25 | — | — |
| BR | — | — | 0.25 | — |
| ER | — | — | — | 0.25 |
| UVAPF (in-vitro) | 14.7 ± 0.8 | 17.98 ± 0.81 | 13.84 ± 0.31 | 13.5 ± 0.89 |
| SPF (in-vitro) | 22.47 ± 0.8 | 27.09 ± 1.93 | 27.44 ± 0.97 | 25.4 ± 1.09 |

The data in table 3 shows that an enhanced UVAPF is obtained when palmitic acid as shown in example 6; or stearic acid as shown in example 7, were used in the composition.

The data in table 3 also shows that an enhanced UVAPF is obtained when BR as shown in example 8; or ER as shown in example 9, were used in the composition.

In conclusion, the sunscreen compositions as per the present invention provide an improved UVAPF.

1. A sunscreen composition comprising:
a. 0.1 to 5 wt % UVA organic sunscreen,
b. 0.1 to 10 wt % 2-ethylhexyl 2-cyano-3,3-diphenylacrylate,
c. 0.1 to 2.5 wt % of a compound selected from resorcinol, phenylethyl resorcinol, 4-alkyl substituted resorcinol and mixtures thereof,
d. 4 to 25 wt % fatty acid; and
e. 0.1 to 10 wt % soap.

2. The sunscreen composition according to claim 1 further comprising a nonionic surfactant having HLB value in the range 9 to 20.

3. The sunscreen composition according to claim 1 wherein the nonionic surfactant is fatty alcohol ethoxylate with saturated carbon chain having HLB in the range 15.5 to 20.

4. The sunscreen composition according to claim 1 further comprising 0.1 to 5 wt % of a polymer.

5. The sunscreen composition according to claim 1 comprising 0.1 to 3 wt % soap.

6. The sunscreen composition according to claim 1 comprising 8 to 20 wt % fatty acid.

7. The sunscreen composition according to claim 1 wherein 4-alkyl-substituted resorcinol is selected from 4-methyl resorcinol, 4-ethyl resorcinol, 4-propyl resorcinol, 4-isopropyl resorcinol, 4-butyl resorcinol, 4-pentyl resorcinol, 4-hexyl resorcinol, 4-heptyl resorcinol, 4-octyl resorcinol and mixtures thereof.

8. The sunscreen composition according to claim 1 wherein 4-alkyl-substituted resorcinol is selected from 4-ethyl resorcinol, 4-hexyl resorcinol and mixtures thereof.

9. The sunscreen composition as per claim 1 further comprising 0.001 to 15 wt % of a skin lightening agent.

10. The sunscreen composition as per claim 1, wherein the UVA sunscreen is selected from 4-tert-butyl-4′-methoxydibenzoylmethane, 2-methyldibenzoylmethane, 4-isopropyldibenzoyl-methane, 4-tert-butyldibenzoylmethane, 2,4-dimethyldibenzoylmethane, 2,5-dimethyldibenzoylmethane, 4,4′-diisopropyl-dibenzoylmethane, 2-methyl-5-isopropyl-4′-methoxydibenzoylmethane, 2-methyl-5-tert-butyl-4′-methoxy-dibenzoyl methane, 2,4-dimethyl-4′-methoxy dibenzoylmethane or 2,6-dimethyl-4-tert-butyl-4′-methoxy-dibenzoylmethane.

11. The sunscreen composition as per claim 1, wherein the UVA sunscreen is 4-tert-butyl-4′-methoxydibenzoylmethane.

12. The sunscreen composition as per claim 1, wherein the composition is a leave-on composition.

13. The sunscreen composition as per claim 1, wherein the composition is a wash-off composition.

14. A method of providing improved UVAPF, wherein the method comprises the steps of:
a. applying on the skin a sunscreen composition as per claim 1; and
b. optionally, rinsing with water.

15. (canceled)
